(19) United States
(12) Patent Application Publication
(10) Pub. No.: US 2022/0392127 A1
(43) Pub. Date: Dec. 8, 2022

(54) IMAGE ANNOTATION METHOD

(71) Applicant: Compal Electronics, Inc., Taipei City (TW)

(72) Inventors: Feng-Yu Liu, Taipei City (TW); Yi-Ching Chen, Taipei City (TW)

(21) Appl. No.: 17/412,257

(22) Filed: Aug. 26, 2021

(30) Foreign Application Priority Data
Jun. 3, 2021 (TW) 110120137

(51) Int. Cl. G06T 11/60; G06T 3/40; G06T 7/00; G06N 5/04

(52) U.S. Cl. CPC G06T 11/60; G06T 3/40; G06T 7/0012; G06N 5/04; G06T 2200/24; G06T 2207/30008; G06T 2207/20081; G06T 2210/41; G16H 30/40

(57) ABSTRACT

An image annotation method for an image annotation system is provided. The image annotation method includes the following steps. Firstly, an original image is provided. Then, an image pre-processing process is performed on the original image to generate an adjusted image. Then, the adjusted image is inferred according to a deep learning model, so that at least one predicted result is obtained. Then, an image post-processing process is performed on the adjusted image and the at least one predicted result to generate a final image. Then, the final image, the at least one predicted result and at least one annotation of the at least one predicted result are displayed.

### FIELD OF THE INVENTION

The present invention relates to an image processing method, and more particularly to an image annotation method.

### BACKGROUND OF THE INVENTION

An image annotation is a process of attaching annotations to images to assist readers in understanding the relevant information in the images. For example, the medical image annotation attaches the images with important information for clinical diagnosis. The annotator needs to analyze the objects in the image and make annotations.

However, the manual process of making image annotations not only requires professional knowledge and judgment in related fields but also takes a lot of time and concentration to identify the annotated objects. In other words, the manual process is costly and inefficient.

Therefore, there is a need of providing an improved image annotation method in order to overcome the drawbacks of the conventional technologies.

### SUMMARY OF THE INVENTION

An object of the present invention provides an image annotation method in order to overcome the drawbacks of the conventional technologies.

Another object of the present invention provides an image annotation method. A trained deep learning model is used to infer the adjusted image and automatically generate annotations in order to provide more accurate predicted results. When compared with the manual process, the labor cost and the time cost of the image annotation method are reduced, and the image annotation task is simplified.

A further object of the present invention provides an image annotation method. The image annotation method allows the user to load images and annotations from the image set. The images with annotations can continuously undergo the image annotation operation according to the deep learning model. Alternatively, the images of the image set having not undergone annotations can undergo the image annotation operation in batch. Consequently, the accuracy of the image annotation can be enhanced, and the operation time can be reduced.

In accordance with an aspect of the present invention, an image annotation method for an image annotation system is provided. The image annotation method includes the following steps. Firstly, an original image is provided. Then, an image pre-processing process is performed on the original image to generate an adjusted image. Then, the adjusted image is inferred according to a deep learning model, so that at least one predicted result is obtained. Then, an image post-processing process is performed on the adjusted image and the at least one predicted result to generate a final image. Then, the final image, the at least one predicated result and at least one annotation of the at least one predicted result are displayed.

In accordance with another aspect of the present invention, an image annotation method for an image annotation system is provided. The image annotation method includes the following steps. Firstly, an image set and an image annotation system are provided. Then, a plurality of images and a plurality of annotations of the image set are loaded. Then, one of the plurality of images is selected as a selected image, and a determining step is performed to determine whether at least one specified annotation of the plural annotations is corresponding to the selected image. When a determining condition of the determining step is satisfied, the at least one specified annotation is loaded as an original annotation. When the determining condition of the determining step is not satisfied, a blank annotation is loaded as the original annotation. Then, the image annotation system acquires the selected image and the original annotation. Then, an image pre-processing process is performed on the selected image to generate an adjusted image. Then, the adjusted image is inferred according to a deep learning model, so that at least one predicted result is generated. Then, an image post-processing process is performed on the adjusted image and the at least one predicted result to generate a final image. Then, the final image, the original annotation, the at least one predicted result and at least one predicted annotation of the at least one predicted result are displayed on a graphical interface. Then, an editing operation is performed on the graphical interface to generate a final annotation.

The above contents of the present invention will become more readily apparent to those ordinarily skilled in the art after reviewing the following detailed description and accompanying drawings, in which:

### BRIEF DESCRIPTION OF THE DRAWINGS

FIG. 1 is a flowchart illustrating an image annotation method according to a first embodiment of the present invention;

FIG. 2 is a schematic diagram illustrating a graphical interface for the image annotation method according to the first embodiment of the present invention;

FIGS. 3A, 3B and 3C are schematic diagrams illustrating the steps of the image pre-processing process in the image annotation method according to the first embodiment of the present invention;

FIGS. 4A and 4B are schematic diagrams illustrating the steps of the image post-processing process in the image annotation method according to the first embodiment of the present invention;

FIG. 5 is a flowchart illustrating an image annotation method according to a second embodiment of the present invention;

FIG. 6 is a flowchart illustrating an image annotation method according to a third embodiment of the present invention; and

FIGS. 7A and 7B illustrate a flowchart of an image annotation method according to a fourth embodiment of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

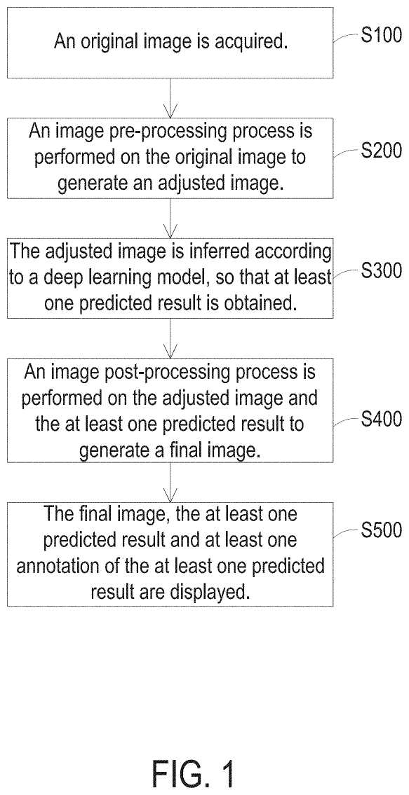

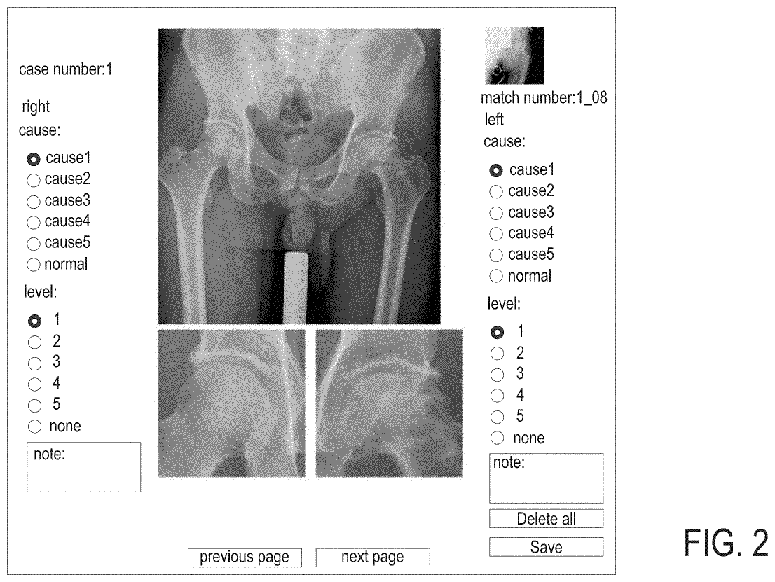

Please refer to FIGS. 1 and 2. FIG. 1 is a flowchart illustrating an image annotation method according to a first embodiment of the present invention. FIG. 2 is a schematic diagram illustrating a graphical interface for the image annotation method according to the first embodiment of the present invention. The image annotation method is applied to an image annotation system. In an embodiment, the image annotation method is a medical image annotation method, and the image annotation system is a medical image annotation system. Especially, the image annotation method is a hip joint image annotation method, and the image annotation system is a hip joint image annotation system. It is noted that the examples of the image annotation method and the image annotation system are not restricted.

The image annotation method of this embodiment includes the following steps.

Firstly, in a step S100, an original image is acquired. For example, the original image is a medical image or a hip joint image. The type of the original image is not restricted. For example, the original image is an ultrasonic image acquired by an ultrasonic device, or the original image is an X-ray film acquired by an X-ray device, or the original image is any other appropriate image acquired by an image pickup device.

Then, in a step S200, an image pre-processing process is performed on the original image to generate an adjusted image.

Then, in a step S300, the adjusted image is inferred according to a deep learning model. Consequently, at least one predicted result is obtained.

Then, in a step S400, an image post-processing process is performed on the adjusted image and the at least one predicted result to generate a final image.

In a step S500, the final image, the at least one predicted result and at least one annotation of the at least one predicted result are displayed. In an embodiment, the final image, the at least one predicted result and the at least one annotation of the at least one predicted result are displayed on a graphical interface (e.g., a graphical interface of a display device) in an overlap display manner. It is noted that the ways of displaying the final image, the at least one predicted result and the at least one annotation of the at least one predicted result are not restricted.

In some embodiments, the image pre-processing process in the step S200 of the image annotation method can be implemented with a processor or a computation unit of the image annotation system. Preferably but not exclusively, the processor (or the computation unit) is a central processing unit (CPU) or a graphic processing unit (GPU). For example, in the image pre-processing process, an image patching operation and an image scaling operation are sequentially performed on the image. Consequently, the size of the adjusted image can meet the input size requirement of the deep learning model. It is noted that the deep learning model used in the image annotation method of the present invention is a deep learning model that has been trained. The model structure of the deep learning model is applied to a Convolutional Neural Network (CNN) model. For example, the deep learning model can be a Region-based Convolutional Neural Networks (R-CNN) model, a You Only Look Once (YOLO) model, a Single-Shot Multibox Detector (SDD) model, a CenterNet model, a Neural Architecture Search (NAS) model, or any other appropriate deep learning model.

Generally, a pre-training method of the deep learning model in the present invention uses the dataset which has been annotated. After the dataset is transferred through a neural network in a forward pass manner and the loss is calculated by using a loss function, a gradient is calculated by using a backpropagation process. Moreover, the parameter is updated according to the calculated result of an optimizer. The calculation process is repeatedly performed until the loss is converged to be in the ideal range. Consequently, the pre-training method of the deep learning model is finished. Moreover, since the deep learning model used in the present invention is trained according to the above pre-training method, the accuracy of the predicted results can be enhanced. When the deep learning model is cooperatively used in the image annotation method of present invention, the labor cost and the time cost are reduced. In other words, the image annotation task can be simplified.

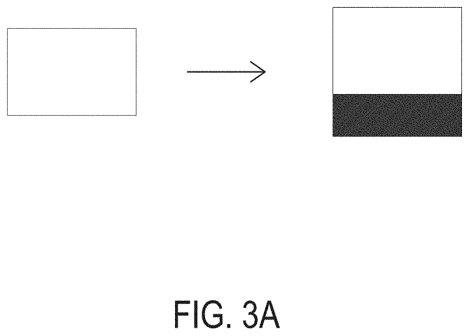

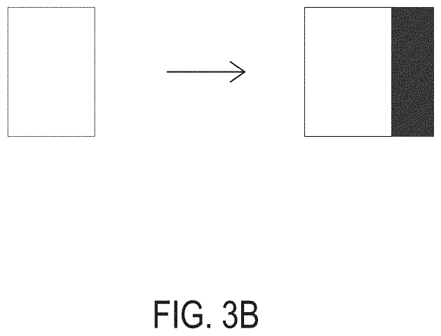

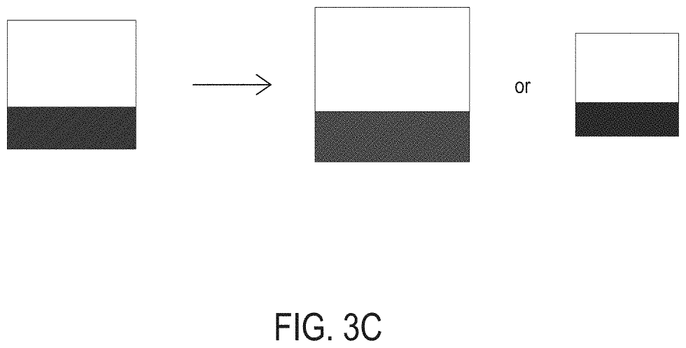

For example, in an example of the image pre-processing process, a square image is required according to the input size of the deep learning model, and the original image is a rectangular image. Please refer to FIGS. 3A, 3B and 3C. FIGS. 3A, 3B and 3C are schematic diagrams illustrating the steps of the image pre-processing process in the image annotation method according to the first embodiment of the present invention. Firstly, an image patching operation is performed on the original image to add 0 values to the short side of the rectangular image. Consequently, the width and the length of the image are equal. For example, as shown in FIG. 3A, pixels are patched along a vertical direction according to the input size requirement. As shown in FIG. 3B, pixels are patched along a horizontal direction according to the input size requirement. Consequently, the rectangular image is converted into the square image. As shown in FIG. 3C, an image scaling operation is performed on the square image. The square image is scaled up or scale down by K times. Consequently, the adjusted image can comply with the input size requirement. For example, if the sizes of the square image after the image patching operation is 200×200 pixels and the input size of the deep learning model is 300×300 pixels, the K value is 1.5. After the adjusted image generated in this step is enlarged by 1.5 times, the size is 300×300 pixels. In some embodiments, the K is a positive value larger than 0.

Please refer to FIG. 1 again. After the adjusted image is inferred according to the deep learning model in the step S300 of the image annotation method, at least one predicted result is generated. The step S300 can be implemented with the processor of the image annotation system. In an embodiment, the predicted result includes a first image part that is possibly needed to be annotated and a second image part image that is needed to be annotated. The first image part and the second image part are predicted according to the combination of the professional application requirement and the deep learning model. In some embodiments, at least one predicted result includes a plurality of predicted results. The plurality of predicted results are displayed at specified locations on the image in a form of a square box or a circular frame, and the actual nouns and probabilities are correspondingly displayed. Alternatively, scores and confidence values are collaboratively displayed. It is noted that the ways of displaying the predicted results are not restricted.

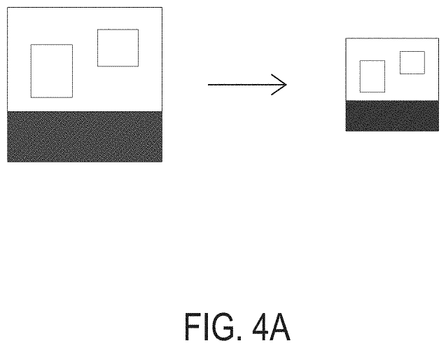

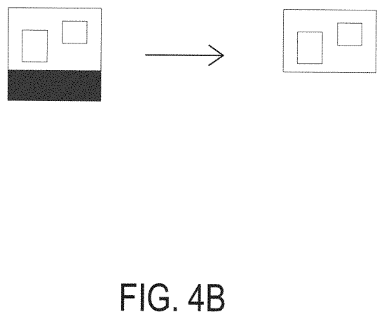

After the at least one predicted result is generated in the step S300, the image post-processing process of the step S400 is performed to generate the final image. The image post-processing process in this step can be implemented with the processor or the computation unit of the image annotation system. Particularly, after the image post-processing is performed and completed, the adjusted image and the at least one predicted result are restored to the size of the original image corresponding to the image pre-processing process. In other words, the sequential image scaling operation and the image restoring operation are performed sequentially. Please refer to FIGS. 4A and 4B. FIGS. 4A and 4B are schematic diagrams illustrating the steps of the image post-processing process in the image annotation method according to the first embodiment of the present invention. In the image post-processing process, the inverse calculation corresponding to the image scaling operation of the image pre-processing process is performed. In other words, the adjusted image is scaled up or scaled down by 1/K times. For example, the adjusted image has a size of 300×300 pixels. After the image scaling operation of the image post-processing process is completed, the adjusted image is scaled down by 1.5 times. That is, the size of the image is reduced to 200×200 pixels. Then, the inverse calculation corresponding to the above image patching operation is performed, and the area of complementing the 0 values is removed. This step is also regarded as an image restoring operation. Consequently, the final image contains annotations, and the size of the final image is the same as the size of the original image. If the original image has not undergone the image patching operation, the action of removing the patched content will be automatically omitted in this step. In other words, after the final image is generated in this step, the final image denotes that the predicted results after the image scaling operation are accurately displayed on the original image.

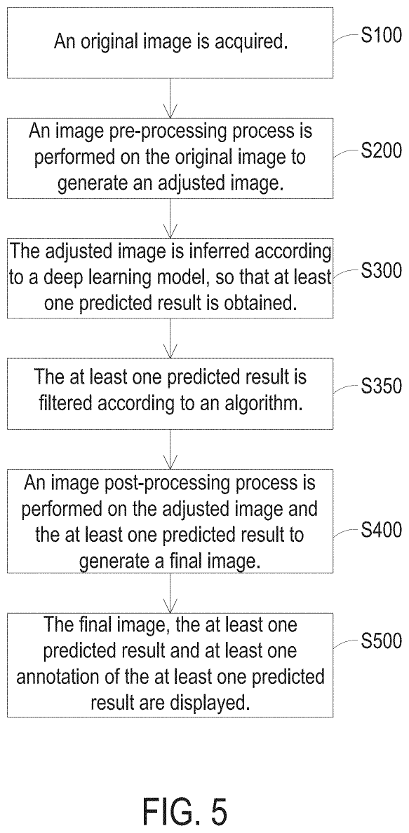

Please refer to FIGS. 4A, 4B and 5. FIG. 5 is a flowchart illustrating an image annotation method according to a second embodiment of the present invention. In some situations, a plurality of predicted results are generated after the step S300. For increasing the accuracy of the image annotation method, the image annotation method of this embodiment further includes a step S350 when compared with the first embodiment. The step S350 is performed after the step S300 and before the step S400. In the step S350, the at least one predicted result is filtered according to an algorithm. Preferably but not exclusively, the algorithm is a Non-Maximum Suppression (NMS) algorithm. In step S400, the image post-processing process is performed on the adjusted image that has been filtered according to the algorithm. Consequently, the final image is generated.

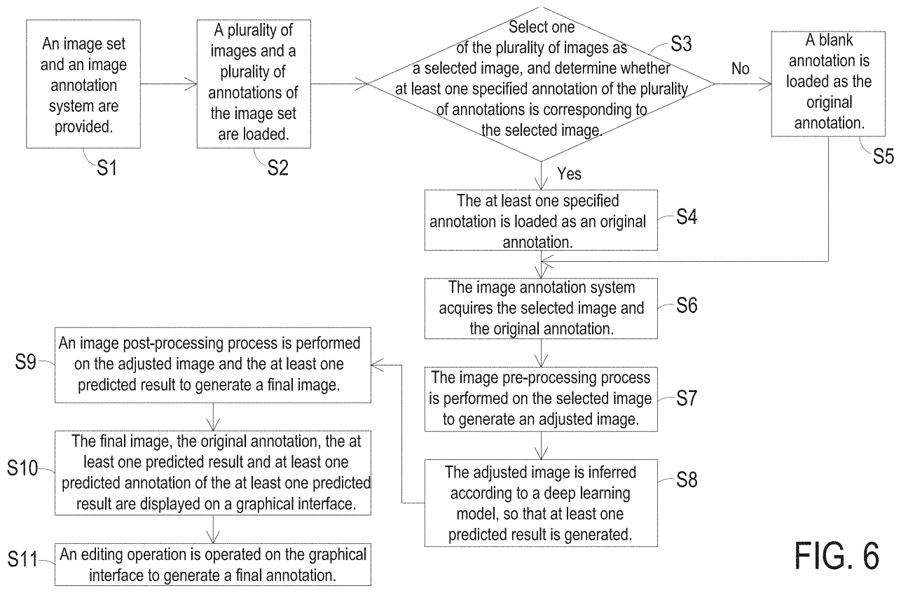

In some embodiments, the present invention provides an image annotation method for the user to select a specified image from an image set. Please refer to FIG. 6. FIG. 6 is a flowchart illustrating an image annotation method according to a third embodiment of the present invention. The image annotation method includes the following steps.

Firstly, in a step S1, an image set and an image annotation system are provided. In an embodiment, the image set is selected by the user. Alternatively, the image set is automatically selected by the image annotation system.

Then, in a step S2, a plurality of images and a plurality of annotations of the image set are loaded. Then, in a step S3, one of the plurality of images is selected as a selected image, and determine whether at least one specified annotation of the plurality of annotations is corresponding to the selected image. In other words, the step S3 is used to determine whether old annotations are corresponding to the selected image.

When the determining condition of the step S3 is satisfied (i.e., at least one specified annotation of the plurality of annotations is corresponding to the selected image), a step S4 is performed after the step S3. In the step S4, the at least one specified annotation is loaded as an original annotation. When the determining condition of the step S3 is not satisfied (i.e., there is no specified annotation of the plurality of annotations corresponding to the selected image), a step S5 is performed after the step S3. In the step S5, a blank annotation is loaded as the original annotation.

Then, in a step S6, the image annotation system acquires the selected image and the original annotation.

Then, in a step S7, the image pre-processing process is performed on the selected image to generate an adjusted image.

Then, in a step S8, the adjusted image is inferred according to a deep learning model. Consequently, at least one predicted result is generated.

Then, in a step S9, an image post-processing process is performed on the adjusted image and the at least one predicted result to generate a final image.

Then, in a step S10, the final image, the original annotation, the at least one predicted result and at least one predicted annotation of the at least one predicted result are displayed on a graphical interface.

Then, in a step S11, an editing operation is operated on the graphical interface to generate a final annotation. The steps S6˜S10 are similar to the steps S100˜S500 in the image annotation method of the first embodiment and not redundantly described herein. In comparison with the step S100 of the first embodiment, the original annotation is additional acquired in the step S6 of this embodiment. In comparison with the step S500 of the first embodiment, the original annotation is additionally displayed on the graphical interface in the step S10 of this embodiment. In the step S11, the editing operation is operated on the graphical interface. Preferably but not exclusively, the step S11 (or the editing operation) is performed by the user. After the editing operation is completed by the user, the final annotation is generated. The final annotation includes the original annotation, a part of the original annotation, or none of the original annotation. In other words, the image annotation method of this embodiment allows the user to load images and annotations from the image set. The images with annotations can continuously undergo the image annotation operation according to the deep learning model. Alternatively, the images of the image set having not undergone annotations can undergo the image annotation operation in batch. Consequently, the accuracy of the image annotation can be enhanced, and the operation time can be reduced.

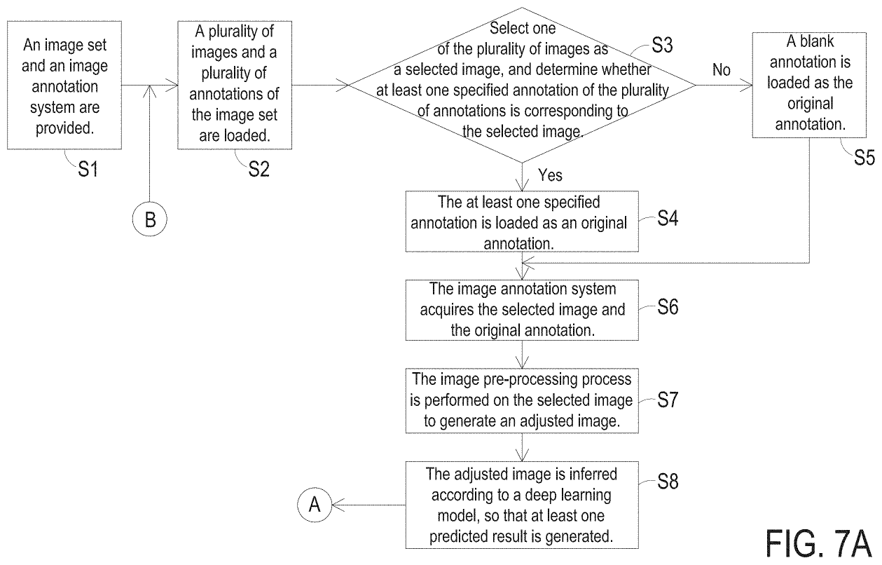

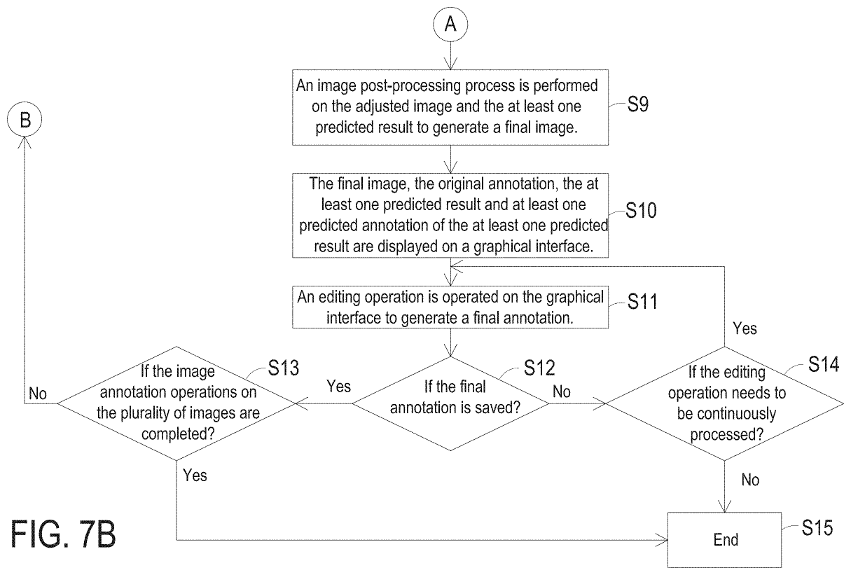

Please refer to FIGS. 7A and 7B. FIGS. 7A and 7B illustrate a flowchart of an image annotation method according to a fourth embodiment of the present invention. The steps S1˜S11 of the annotation method of the fourth embodiment are similar to the steps S1˜S11 of the third embodiment as shown in FIG. 6. In comparison with the third embodiment, the annotation method of the fourth embodiment further includes steps S12˜S15 after the step S11.

After the step S11, a step S12 is performed to determine whether the final annotation is saved.

When the determining condition of the step S12 is satisfied (i.e., the final annotation is saved), a step S13 is performed to determine whether the image annotation operations on the plurality of images are completed.

When the determining condition of the step S12 is not satisfied (i.e., the final annotation is not saved), a step S14 is performed to determine whether the editing operation needs to be continuously processed.

When the determining condition of the step S13 is satisfied (i.e., the image annotation operations on the plurality of images are completed), a step S15 is performed. In the step S15, the image annotation method is ended. When the determining condition of the step S13 is not satisfied (i.e., the image annotation operations on the plurality of images are not completed), the step S2 is repeatedly processed and the steps after the step S2 are performed sequentially.

When the determining condition of the step S14 is satisfied (i.e., the editing operation needs to be continuously processed), the editing operation of the step S11 is performed again, and the steps after the step S11 are performed sequentially. When the determining condition of the step S14 is not satisfied (i.e., the editing operation needn't be continuously processed), the step S15 is performed. Consequently, the image annotation method is ended.

In some embodiments, the determining processes of the steps S12˜S14 are implemented through the interaction between the user and the graphical interface. For example, the image annotation system inquires whether the user intends to save the final annotations through the graphical interface, whether the user completes the image annotation operations on the plurality of images, or whether the user intends to continuously perform the editing operation. The user can respond to the graphical interface in a touch control manner, a voice control manner, a keyboard control manner or a mouse control manner, but not limited thereto.

From the above descriptions, the present invention provides the image annotation method. The annotation can be automatically inferred and generated according to the deep learning model. Consequently, the accuracy of the predicted results can be enhanced, and the labor cost and the time cost are reduced. In other words, the image annotation task can be completed easily. Moreover, the image annotation method of this embodiment allows the user to load images and annotations from the image set. The images with annotations can continuously undergo the image annotation operation according to the deep learning model. Alternatively, the images of the image set having not undergone annotations can undergo the image annotation operation in batch. Consequently, the accuracy of the image annotation can be enhanced, and the operation time can be reduced.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An image annotation method for an image annotation system, the image annotation method comprising steps of:
(a) acquiring an original image;
(b) performing an image pre-processing process on the original image to generate an adjusted image;
(c) inferring the adjusted image according to a deep learning model, so that at least one predicted result is obtained;
(d) performing an image post-processing process on the adjusted image and the at least one predicted result to generate a final image; and
(e) displaying the final image, the at least one predicted result and at least one annotation of the at least one predicted result.

2. The image annotation method according to claim 1, wherein when the image pre-processing process is performed, an image patching operation and an image scaling operation are performed on the original image sequentially, so that a size of the adjusted image matches an input size requirement of the deep learning model.

3. The image annotation method according to claim 2, wherein when the image patching operation is performed, pixels are patched along a vertical direction or a horizontal direction of the original image according to the input size requirement of the deep learning model.

4. The image annotation method according to claim 1, wherein after the image post-processing is performed, the adjusted image and at least one predicted result are restored to a size of the original image corresponding to the image pre-processing process.

5. The image annotation method according to claim 1, wherein after the step (c) and before the step (d), the image annotation method further comprises a step of filtering the at least one predicted result according to an algorithm.

6. The image annotation method according to claim 5, wherein the algorithm is a Non-Maximum Suppression (NMS) algorithm.

7. The image annotation method according to claim 1, wherein when the image post-processing is performed, the adjusted image and the at least one predicted result undergo an image scaling operation and an image restoring operation sequentially.

8. The image annotation method according to claim 1, wherein the final image, the at least one predicted result and the at least one annotation of the at least one predicted result are displayed on a graphical interface in an overlap display manner.

9. An image annotation method for an image annotation system, the image annotation method comprising steps of:
(a) providing an image set and an image annotation system;
(b) loading a plurality of images and a plurality of annotations of the image set;
(c) selecting one of the plurality of images as a selected image, and determining whether at least one specified annotation of the plurality of annotations is corresponding to the selected image;
(d) when a determining condition of the step (c) is satisfied, loading the at least one specified annotation as an original annotation;
(e) when the determining condition of the step (c) is not satisfied, loading a blank annotation as the original annotation;
(f) the image annotation system acquiring the selected image and the original annotation;
(g) performing an image pre-processing process on the selected image to generate an adjusted image;
(h) inferring the adjusted image according to a deep learning model, so that at least one predicted result is generated;
(i) performing an image post-processing process on the adjusted image and the at least one predicted result to generate a final image;
(j) displaying the final image, the original annotation, the at least one predicted result and at least one predicted annotation of the at least one predicted result on a graphical interface; and
(k) performing an editing operation on the graphical interface to generate a final annotation.

10. The image annotation method according to claim 9, wherein after the step (k), the image annotation method further comprises steps of:
(l) determining whether the final annotation is saved;
(m) determining whether the image annotation operations on the plurality of images are completed;
(n) determining whether the editing operation is continuously processed; and
(o) ending the image annotation method,
wherein when a determining condition of the step (l) is satisfied, the step (m) is performed after the step (l), when the determining condition of the step (l) is not satisfied, the step (n) is performed after the step (l);
wherein when a determining condition of the step (m) is satisfied, the step (o) is performed after the step (m), when the determining condition of the step (m) is not satisfied, the step (b) is performed again after the step (m);
wherein when a determining condition of the step (n) is satisfied, the step (k) is performed again after the step (n), when the determining condition of the step (n) is not satisfied, the step (o) is performed after the step (n); and
wherein the step (k) is performed by a user, and the step (l), the step (m) and the step (n) are implemented through an interaction between the user and the graphical interface.

11. The image annotation method according to claim 9, wherein when the image pre-processing process is performed, an image patching operation and an image scaling operation are performed on the selected image sequentially, so that a size of the adjusted image matches an input size requirement of the deep learning model.

12. The image annotation method according to claim 11, wherein when the image patching operation is performed, pixels are patched along a vertical direction or a horizontal direction of the selected image according to the input size requirement of the deep learning model.

13. The image annotation method according to claim 9, wherein after the image post-processing is performed, the adjusted image and the at least one predicted result are restored to a size of the selected image corresponding to the image pre-processing process.

14. The image annotation method according to claim 9, wherein after the step (h) and before the step (i), the image annotation method further comprises a step of filtering the at least one predicted result according to an algorithm.

15. The image annotation method according to claim 14, wherein the algorithm is a Non-Maximum Suppression (NMS) algorithm.

16. The image annotation method according to claim 9, wherein when the image post-processing is performed, the adjusted image and the at least one predicted result undergo an image scaling operation and an image restoring operation sequentially.

17. The image annotation method according to claim 9, wherein the final image, the original annotation, the at least one predicted result and the at least one annotation of the at least one predicted result are displayed on the graphical interface in an overlap display manner.
